(19) United States
(12) Patent Application Publication
(10) Pub. No.: US 2004/0101551 A1
(43) Pub. Date: May 27, 2004

(54) Transdermal therapeutic system for releasing venlafaxine

(76) Inventor: Thorsten Selzer, Neuwied (DE)

Correspondence Address: BIRCH STEWART KOLASCH & BIRCH, PO BOX 747, FALLS CHURCH, VA, 22040-0747, US

(21) Appl. No.: 10/362,915

(22) PCT Filed: Aug. 18, 2001
(86) PCT No.: PCT/EP01/09531

(30) Foreign Application Priority Data
Aug. 30, 2000 (DE) 100 42 4120

(51) Int. Cl. A61K 9/70

(52) U.S. Cl. 424/449

(57) ABSTRACT

A transdermal therapeutic system in the form of a patch for administration of the active agent venlafaxine comprises an active agent-impermeable backing layer, an active substance reservoir connected therewith and containing the active agent venlafaxine, a pressure-sensitive adhesive layer on the skin side, and an active agent-impermeable protective layer detachable prior to application.

The present invention relates to transdermal therapeutic systems comprising the active substance venlafaxine and enabling the release of said active substance via the skin. The invention furthermore comprises the therapeutic use of such systems for various medical indications.

Venlafaxine is a serotonine and noradrenaline reuptake inhibitor and is used in the therapy and prophylaxis of depressions and states of anxiety.

Oral application of venlafaxine does, however, involve a number of drawbacks which are caused by pharmacokinetics. In the initial phase after oral administration, i.e. after approx. 2 to 4 h, “plasma peaks” occur, that is, the plasma concentration of venlafaxine reaches relatively high values for a short period. With a usual single dose of 25 mg, this can lead to unwanted side effects.

On the other hand, venlafaxine is metabolised and excreted relatively quickly. Its elimination half-life is in the range of 3 to 5 h and is thus rather short. This leads to a relatively quick decrease in its action so that 2 to 3 successive single applications per day will be necessary. These disadvantages must be taken into consideration particularly in the light of a long-term therapy, for example in the case of depressions.

Transdermal therapeutic systems (TTS) are administration forms which are applied to the skin and which release an active substance to the skin, in this manner making the active substance systemically available.

According to the state of the art, TTS consist of a carrier layer impermeable to the medicinal agent (also called backing layer), a medicinal agent-containing reservoir layer, as well as a pressure-sensitive adhesive layer for attaching the system on the skin. The latter layer may also be identical with the medicinal agent-containing layer. As a rule, TTS also have a likewise active agent-impermeable backing layer, which is to be removed prior to application. In addition, other components may be present such as, for example, a control membrane limiting the active substance release. The medicinal agent-containing reservoir layer mostly consists of polymer base substances; it may furthermore contain various auxiliary or additive substances.

It was the object of the present invention to provide an administration form for the active substance venlafaxine the use of which will avoid the disadvantages involved in the oral administration of venlafaxine described above. In addition, the said administration form should enable a sufficiently high active agent flow in vivo.

Furthermore, it is to be demanded that it should be possible to produce such an administration form by means of common manufacturing methods in a cost-effective manner.

Surprisingly, this object is achieved by a transdermal therapeutic system in the form of a patch according to Claim 1 as well as by the embodiments described in the subclaims.

Consequently, the invention comprises TTS in patch form for administration of the active agent venlafaxine which have an active agent-impermeable backing layer, an active agent reservoir connected thereto and containing the active agent venlafaxine, a pressure-sensitive adhesive layer on the skin side, and an active agent-impermeable protective layer which is detachable prior to application.

The TTS according to the invention enable, over a prolonged period of application, a constant release of the active agent venlafaxine to and through the skin, whereby this active substance becomes systemically available. In this way, the relatively quick elimination of venlafaxine can be compensated by continual further delivery from the active substance reservoir of the TTS, and the therapeutic value of the administration of the medicinal agent is increased. This is of advantage, in particular, in the long-term therapy of depressions. As compared to oral administration forms, a continued therapeutic action can be achieved with a relatively low application frequency.

Further advantages are based on the fact that in the transdermal administration the metabolism of the active agent during the first intestine-liver passage (“first-pass” effect) is prevented, thus increasing the half-life. In addition, in this manner it is possible to avoid problems such as gastrointestinal intolerance or insufficient enteral absorption.

The TTS according to the invention can be manufactured both in the form of matrix systems and in the form of bag- or pouch-reservoir systems, respectively membrane systems.

The term “matrix systems” does not only include such systems as contain the active agent dissolved in a layer-shaped synthetic resin or plastics matrix and release the same from this matrix, but also such systems as contain the active agent adsorbed to fibre material such as, for example, cotton woven fabric or cotton nonwoven. This fibre material may be embedded in a synthetic resin or a plastics matrix.

Basically a large number of polymers, resins and additives can be used for producing the active substance reservoir or the active substance matrix provided that the substances coming into contact with the skin are well tolerated by the skin, and as long as the formulation prepared therewith is capable of releasing the active agent venlafaxine to the skin.

The active substance reservoir of the TTS according to the invention may furthermore contain various auxiliary and additive substances, for example from the group of the solubilizers, solvents, plasticizers, permeation enhancers, pH regulators, antioxidants and preservatives.

In the production of the TTS according to the invention, in the simplest case one may proceed by coarsely, colloidally or molecularly dispersing venlafaxine in a solution of matrix base polymers and coating the mixture on a suitable support, for example a thermoplastic film provided with a silicone layer. After drying and evaporating the solvent portions, the active substance-containing matrix layer is covered with a further film which later constitutes the backing layer of the TTS. From such a laminate, TTS are produced by punching flat-shaped pieces in the desired geometric shape and size.

Suitable base polymers for the active substance matrix and the pressure sensitive adhesive layer are polyacrylates, poly(meth)acrylates, polyacrylic acid, cellulose derivatives, especially methyl and ethyl celluloses, isobutylene, ethylene vinyl acetate, natural and synthetic rubbers such as styrene-diene copolymers, styrene-butadiene block copolymers, isoprene block copolymers, acrylonitrile-butadiene rubber, butyl rubber or neoprene rubber, as well as hot-melt adhesives. Suitable pressure sensitive adhesives are also those based on silicone. With advantage, suitable mixtures of the polymers mentioned may be used too.

The term “hot-melt adhesives” comprises any adhesives which are liquefied not by solvents, but by melting at elevated temperature, for example in the range of 60-200° C. Mixtures of esters of the hydrogenated colophony with cellulose derivates are suitable for use as hot-melt adhesives.

Apart from the polymers mentioned, further base polymers known to those skilled in the art may be used for producing the matrix or the pressure sensitive adhesive layer, provided that they are compatible with the active agent venlafaxine.

A particularly preferred embodiment of the invention is characterized in that the active agent venlafaxine is present in the TTS in combination with a solubilizer, preferably in dissolved state; a mixture of solubilizers may be used as well.

Examples of preferred solubilizers are polyvalent alcohols such as 1,2-propanediol, the various butanediols, glycerol, polyethylene glycol 400, tetrahydrofurfuryl alcohol, diethylene glycol monoethyl ether, diethyl toluamide and monoisopropylidene glycerol. Especially preferred is the use of 1,2-propanediol. Some of the solubilizers, such as, for example, the 1,2-propanediol, can in addition have permeation enhancing action.

It has turned out to be of advantage for the portion of the solubilizers to be between 1 and 50%-wt, preferably between 5 and 35%-wt, relative to the complete TTS in its final state after manufacture.

To achieve a high active agent flux through the skin, it has, especially in the case of matrix systems, turned out to be advantageous to add one or more permeation-enhancing substances to the matrix, in a constituent amount of 0.1 to 25%-wt, preferably of 1 to 10%-wt, in each case relative to the total weight of the active substance matrix.

As permeation-enhancing substances are suitable, above all, those from the groups of fatty alcohols, fatty acids, polyoxyethylene fatty alcohol ethers, polyoxyethylene fatty acid esters, fatty alcohol esters and fatty acid esters, especially sorbitane monolaurate or esters of long-chain fatty acids with methyl, ethyl or isopropyl alcohol, or esters of fatty alcohols with acetic acid or lactic acid. Substances like oleic acid diethanolamine are also suitable. Especially preferred are polyoxylauryl ethers (Brij®).

According to an especially preferred embodiment, the active substance matrix of the TTS according to the invention may also have a two- or multilayer structure, i.e. it may consist of two or more matrix layers. In this case, the various matrix layers may differ in terms of their composition or in terms of the concentration of the components contained therein. For example, the various matrix layers may have a differing polymer composition, or consist of differing pressure sensitive adhesives. Furthermore, the individual matrix layers may contain different concentrations of active agent or additives such as permeation-enhancing agents, solubilizers and plasticizers. Depending on the application purpose intended, it is possible to adjust, for example, the concentration of these ingredients, especially the active agent concentration, in these layers such that, from the inner layer towards the layer located on the skin side, they become smaller or greater, depending on whether a particular long-term action or a particularly strong initial action is desired.

Further embodiments of the TTS according to the invention provide for the addition, to the active agent matrix or to individual layers of this matrix, of usual plasticizers in a concentration of up to 30%-wt, with particular preference in a concentration of 5-20%-wt, in each case relative to the active substance matrix.

As plasticizers, those from the group of the carbohydrates, alcohols, carboxylic acids, derivatives of carboxylic acids, ethers, esters, and amines may be used with preference.

To achieve a high release rate, it is preferred that a high active agent concentration be provided in the active agent-containing layer(s); however, in this case it has to be taken into consideration that active agent concentrations which are too high may impair the physical stability of the active substance matrix. Therefore, with the TTS according to the invention, active agent concentrations in the range of 0.1 to 50%-wt, preferably in the range from 1 to 10%-wt. are used, in each case related to the total mass of the active substance-containing layer(s).

To make a control of the active agent release possible—if not brought about by other mechanisms—the active substance reservoir may also be provided, on the release side, with a control membrane which has a limited permeability for the active agent and controls the release of the active agent to the skin.

The pressure-sensitive adhesive attachment of the TTS according to the invention on the skin may be accomplished in various ways. For example, the active agent-containing matrix itself may consist of a pressure-sensitive adhesive and thus bring about the connection with the skin, or there is arranged a separate pressure-sensitive adhesive layer which takes over this function. Especially in the case of systems which are—as described above—provided with a skin-facing, non-adhesive control membrane, it is advantageous to accomplish the attachment to the skin by a margin of adhesive surrounding the membrane surface via which the active agent is released to the skin, but not touching the same; i.e. the adhesive margin is not in contact with the control membrane.

The invention further comprises embodiments wherein the venlafaxine-containing active agent reservoir is formed as a bag- or pouch-shaped reservoir filled with a flowable, highly viscous, semi-solid or gel-like matrix containing the active agent. For example, this may be a polymer matrix, especially a plastics matrix, or a solution thereof. It is of particular advantage if the active agent reservoir contains a gelatinizing agent.

In this case, the pouch rear side, which is averted from the skin, has to be impermeable to the active agent, and the side facing the skin (release side) has to be permeable to the active agent. If necessary, an active substance-permeable membrane may take over the function of controlling the release of active agent. Suitable materials for the manufacture of the pouch wall, respectively the control membrane, are known to those skilled in the art.

The TTS according to the invention, apart from the active agent reservoir also have an active agent-impermeable backing layer, as well as a likewise active agent-impermeable, detachable protective layer or stripping film.

Suitable materials for the backing layer are, above all, polyesters which are characterized by their special strength such as, for example, polyethylene terephthalate and polybutylene terephthalate, but also almost any other skin-tolerated plastics, such as polyvinyl chloride, ethylene-vinyl acetate copolymers, polyvinyl acetate, polyethylene, polypropylene, polyurethane, cellulose derivatives and many others. In the individual case, the backing layer may be provided with an additional layer, e.g. by vapour-deposition with metals, especially aluminium.

For the detachable protective layer, basically the same materials may be used as for the backing layer, provided that it has been rendered detachable by a suitable surface treatment such as, for example, siliconization. Other detachable protective layers such as, for example, polytetrafluoroethylene-treated paper, or cellophane® (cellulose hydrate) may, however, also be used.

The TTS according to the invention, containing the active agent venlafaxine, are advantageously suitable for the prophylaxis and therapy of psychoses, especially depressions, as well as of anxiety states, neuroses and psychopathies. In these cases it is of particular advantage that with this administration form a duration of action of at least about one day up to about 7 days can be achieved, depending on the active agent content and the configuration of the control mechanism controlling the active agent release behaviour.

According to a further embodiment of the invention, it is provided that the active substance reservoir contains venlafaxine in combination with at least one further pharmaceutical active agent; preferably this is a centrally active medicinal agent.

The invention will be illustrated by the following Example.

### EXAMPLE

The TTS according to the invention may, for example, be manufactured as follows:

50 g of venlafaxine as well as 20 g of a suitable permeation-enhancing substance (e.g. Brij®30) are dissolved in 200 g of 1,2-propanediol. This solution is introduced in a silicon adhesive (No. 4301; by Dow Corning, USA) and dispersed by a suitable stirring apparatus, so that a liquid-liquid dispersion is obtained that is as homogenous as possible. This dispersion is homogenously coated onto a carrier film, e.g. of polyethylene terephthalate, using a suitable device. Subsequently, by controlled drying, the solvent of the silicon adhesive as well as possible portions of the propanediol are removed. Controlled drying means that the coated laminate is subjected to a particular drying temperature, drying rate, or drying time in order to set the intended content of the volatile substances (e.g. solubilizers).

The laminate thus obtained is subsequently laminated with a further film of polyethylene terephthalate. Finally, TTS having a certain surface area are punched out and packaged in suitable packing material.

1. Transdermal therapeutic system in patch form for administering the active agent venlafaxine, which comprises an active agent-impermeable backing layer, an active agent reservoir connected therewith and containing the active agent venlafaxine, a pressure-sensitive adhesive layer on the side of the skin, and an active agent-impermeable protective layer detachable prior to application.

2. Transdermal therapeutic system according to claim 1, characterized in that the active agent reservoir is formed as an active agent-containing matrix, the said matrix being a synthetic resin or plastics matrix containing as base polymer(s) one or more polymers which are preferably selected from the group comprising the polyacrlates, poly(meth)acrylates, polyacrylic acids, cellulose derivatives, isobutylene, ethylene vinyl acetate, natural and synthetic rubbers such as styrene-diene copolymers, styrene-butadiene block copolymers, isoprene block copolymers, acrylonitrile-butadiene rubber, butyl rubber or neoprene rubber, as well as hot-melt adhesives.

3. Transdermal therapeutic system according to claim 1 or 2, characterized in that the active substance reservoir is formed as active agent-containing matrix wherein the active agent venlafaxine is present adsorbed on a fibre material, preferably on cotton fabric or nonwoven, said fibre material preferably being embedded in a plastics or a synthetic resin matrix.

4. Transdermal therapeutic system according claim 1, characterized in that the active substance reservoir is formed as a pouch-shaped reservoir containing a high-viscous or semi-solid plastics matrix or a solution thereof, or a gelatinizing agent, or a gel, wherein the active substance venlafaxine is dissolved or dispersed.

5. Transdermal therapeutic system according to one or more of claims 1 to 4, characterized in that the active substance reservoir is provided on the skin-side with an active substance-permeable control membrane or with a control membrane limiting the active agent release rate.

6. Transdermal therapeutic system according to claim 5, characterized in that it comprises an adhesive margin which is adjacent to the outer edge of the membrane surface, is pressure-sensitive adhesive on the skin, and which is not in contact with the control membrane.

7. Transdermal therapeutic system according to one or more of the preceding claims, characterized in that the pressure-sensitive adhesive layer contains, as base polymers, polymers which are preferably selected from the group comprising polyacrylates, poly(meth)acrylates, polyacrylic acid, cellulose derivatives, isobutylene, ethylene vinyl acetate, natural and synthetic rubbers such as styrene-diene copolymers, styrene-butadiene block copolymers, isoprene block copolymers, acrylonitrile-butadiene rubber, butyl rubber or neoprene rubber, as well as hot-melt adhesives and silicone-based pressure-sensitive adhesive.

8. Transdermal therapeutic system according to one or more of the preceding claims, characterized in that the active agent reservoir is formed as a pressure-sensitive adhesive layer.

9. Transdermal therapeutic system according to one or more of the preceding claims characterized in that the active agent reservoir and/or the pressure-sensitive adhesive layer contains at least one auxiliary or additive substance which is selected from the group comprising solvents, solubilizers, plasticizers, permeation enhancers, pH-regulators, antioxidants and preservatives.

10. Transdermal therapeutic system according to one or more of the preceding claims, characterized in that it contains the active agent venlafaxine in combination with a solubilizer, especially preferred a solubilizer with permeation-enhancing action, said active agent preferably being present in dissolved form.

11. Transdermal therapeutic system according to one or more of the preceding claims, characterized in that it contains one or more solubilizers, preferably selected from the group comprising polyvalent alcohols, 1,2-propanediol, butanediol, glycerol, polyethylene glycol 400, tetrahydrofurfuryl alcohol, diethylene glycol monoethyl ether, diethyl toluamide and monoisopropylidene glycerol, the concentration of the solubilizer(s) being between 1 and 50%-wt., preferably between 5 and 35%-wt., relative to the total system.

12. Transdermal therapeutic system according to one or more of the preceding claims, characterized in that it contains one or more permeation-enhancing substances, preferably in a concentration of 0.1 to 25%-wt., especially preferred in a concentration of 1 to 10%-wt., in each case relative to the total system.

13. Transdermal therapeutic system according to claim 12, characterized in that the permeation-enhancing substances are selected from the group comprising fatty alcohols, fatty acids, polyoxyethylene fatty alcohol ethers, polyoxyethylene fatty acid esters, fatty acid esters and fatty alcohol esters.

14. Transdermal therapeutic system according to one or more of the preceding claims, characterized in that the active agent matrix contains one or more plastics, preferably in a concentration of up to 30%-wt, especially preferred in a concentration of 5-20%-wt., in each case relative to the active substance matrix, said softeners preferably being selected from the group comprising carbohydrates, alcohols, carboxylic acids, derivatives of carboxylic acids, ethers, esters and amines.

15. Transdermal therapeutic system according to one or more of the preceding claims, characterized in that the active agent reservoir has a laminar structure with two or more matrix layers which differ in terms of their polymer content and/or their active agent content and/or in terms of the kind and concentration of the permeation enhancers, plasticizers or solubilizers contained therein.

16. Transdermal therapeutic system according to one or more of the preceding claims, characterized in that the active agent concentration is in the range of 0.1 to 50% wt., preferably in the range of 1 to 10%-wt., in each case relative to the total mass of the active agent-containing layer(s).

17. Transdermal therapeutic system according to one or more of the preceding claims, characterized in that the active agent reservoir contains, apart from venlafaxine, at least one further active agent, preferably a centrally active agent.

18. Use of a transdermal therapeutic system containing the active agent venlafaxine for the prophylaxis and therapy of psychoses, especially depressions, as well as of anxiety states, neuroses and psychopathies.

19. Use of a transdermal therapeutic system according to any one of claims 1 to 17, for the prophylaxis and therapy of psychoses, especially depressions, as well as of anxiety states, neuroses and psychopathies.

20. Use of venlafaxine for the production of a transdermal therapeutic system for the prophylactic or therapeutic treatment of patients suffering from psychoses, especially depressions, or anxiety states, neuroses or psychopathies.
